(19) United States
(12) Patent Application Publication
(10) Pub. No.: US 2026/0079161 A1
(43) Pub. Date: Mar. 19, 2026

(54) METHODS FOR DETECTING EXTRACELLULAR BACTERIA

(71) Applicant: IDEXX Laboratories, Inc., Westbrook, ME (US)

(72) Inventors: Jeremy Hammond, Standish, ME (US); Heidi Peta, Edmonton (CA)

(73) Assignee: IDEXX Laboratories, Inc., Westbrook, ME (US)

(21) Appl. No.: 19/330,210

(22) Filed: Sep. 16, 2025

Related U.S. Application Data
(60) Provisional application No. 63/696,082, filed on Sep. 18, 2024.

(51) Int. Cl. G01N 33/58; G01N 1/38; G01N 33/50

(52) U.S. Cl. CPC G01N 33/582; G01N 1/38; G01N 33/5005; G01N 2001/383; G01N 2333/195

(57) ABSTRACT

Disclosed herein are methods and systems for evaluating an aspirated fluid for the presence of extracellular bacteria. The method generally includes receiving a sample cartridge comprising a sample mixture, the sample mixture comprising a liquid biological sample from a subject and a sterilized diluent, in an interior volume of the sample cartridge; imaging the sample mixture with a microscopy system; determining whether bacteria present within the sample mixture is above a configurable threshold; and in response to determining that the bacteria within the sample mixture is above the configurable threshold, determining that the liquid biological sample has extracellular bacteria.

### CROSS REFERENCE TO RELATED APPLICATION

This application claims priority to U.S. Provisional Application Ser. No. 63/696,082, filed Sep. 18, 2024, the entire contents of which are incorporated herein by reference.

### TECHNICAL FIELD

The present disclosure relates to the detection of bacteria in a liquid biological sample, and more specifically to the detection of extracellular bacteria.

### BACKGROUND

Identifying an infection within a lesion can rely on analysis of aspirated samples for the presence of intracellular bacteria. Traditional Fine Needle Aspiration (FNA) glass slide preparations involve aspirating material from a lesion and dispensing the aspirated sample onto a glass slide. The sample is then dried and stained using a series of stains to identify microorganisms. The dried and stained slide is evaluated under a microscope, typically by a clinical pathologist, who provides an interpretation of the cytological findings.

The process of preparing the glass slide impacts the cells, causing them to flatten on the glass surface. This flattening distorts the cells from their natural state. Instead of maintaining their generally spherical shape, the cells are flattened into a two-dimensional form. As a result, the cytoplasm presents a larger surface area along the slide, allowing easier visualization of the nucleic and cytoplasmic components. This also provides space for bacteria, if present, to be visible within the cytoplasm. For example, when the cell is in a natural state, intracellular bacteria (i.e., bacteria positioned within the cell) can be at various positions within the cell, but are not easily viewable as the bacteria can be easily obscured by other cellular components. By contrast, when the cell is presented in a two-dimensional form on a slide, the intracellular bacteria is easier to see because the cellular components are dispersed, and therefore less able to obscure the bacteria. With sufficient staining and optical magnification, a clinical pathologist can positively identify intracellular bacteria.

The presence of intracellular bacteria is an indicator of infection within a lesion, signifying that immune cells, such as neutrophils, have commenced the process of phagocytosis on the bacteria. Conversely, the observation of extracellular bacteria in an FNA sample using conventional methods can result from contamination of the sample rather than true infection. Contamination may occur during sample aspiration if bacteria from the skin surface are transferred to the needle, or during staining preparation, where stains used for multiple samples can become contaminated and subsequently transfer bacteria to any slide stained. This potential for contamination underscores the necessity of identifying intracellular bacteria on the glass slide to confidently diagnose an infected lesion. Because it is often unclear what the source of extracellular bacteria in a FNA sample is (e.g., whether the bacteria are from contamination or are from infection), the detection of intracellular bacteria is the preferred method for identifying infection.

However, slide preparation is time consuming, must be performed by trained users, and cells can be damaged if prepared improperly. For example, cells can be damaged or pushed outside of the field of view during the slide preparation, reducing the likelihood of accurate identification of cell types and conditions. Moreover, many veterinary clinics may infrequently see subjects that present with conditions requiring FNA analysis. As such, even in clinics that have staff trained to prepare FNA slides, the staff may have minimal practical experience. Accordingly, a need to utilize a slide-free process to visualize aspirated tissues or cells in a free-fluid state (i.e., in a three-dimensional volume as compared to a two-dimensional slide) exists. Slide-free processes can include placing the sample fluid in a three-dimensional cavity of a cartridge and visualizing the sample fluid in the cartridge with a microscope. By observing the sample fluid in a free-fluid state, the risk of cell damage and other issues stemming from slide preparation are reduced, reducing the time and skill level required to prepare the sample for evaluation.

However, when presented in a free-fluid state, intracellular bacteria may be difficult to visualize compared with presentation on a two-dimensional slide. By residing in the interior of the cell, intracellular bacteria can be obscured by cytoplasm and other cellular components when viewed in a microscope and can be difficult to identify.

As such, when viewing aspirated fluids in a free-fluid state, extracellular bacteria may be a more reliable indicator of infection. However, as noted above, extracellular bacteria can be introduced to a sample via contamination, and it is difficult to discern whether the presence of extracellular bacteria is from contamination or infection. Accordingly, a need exists for methods and techniques to enable evaluation of extracellular bacteria that is indicative of infection as compared to extracellular bacteria that is indicative of contamination.

### SUMMARY

In one embodiment, the present disclosure relates to a method for evaluating an aspirated fluid. The method includes receiving a cartridge comprising a sample mixture, the sample mixture comprising a liquid biological sample from a subject and a sterilized diluent, in an interior volume of the cartridge; imaging the sample mixture with a microscopy system; determining whether bacteria present within the sample mixture is above a configurable threshold; and in response to determining that the bacteria within the sample mixture is above the configurable threshold, determining that the liquid biological sample has extracellular bacteria.

In another embodiment, the present disclosure relates to a method of detecting extracellular bacteria. The method includes obtaining a liquid biological sample comprising cells from a subject; diluting the liquid biological sample in a sterilized diluent comprising an imaging agent to form a sample mixture; imaging the sample mixture using a microscopy system, wherein the cells remain in a spherical state; and detecting whether extracellular bacteria is present in the liquid biological sample by detecting extracellular bacteria in the sample mixture above a threshold value.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 schematically depicts an example microscopy device, according to one or more embodiments shown and described herein;

FIG. 2 schematically depicts example components of a controller of the microscopy device of FIG. 1, according to one or more embodiments shown and described herein;

FIG. 3 schematically depicts example imaging modes using the microscopy device of FIG. 1, according to one or more embodiments shown and described herein;

FIG. 4 depicts a flowchart of an example method of evaluating an aspirated fluid to detect bacteria, according to one or more embodiments shown and described herein; and

FIG. 5 depicts a flowchart of an example method of the detecting extracellular bacteria in a liquid biological, according to one or more embodiments shown and described herein.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided herein.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, concentration or percentage, or the like, is meant to encompass variations of in some embodiments±20%, in some embodiments±10%, in some embodiments ±5%, in some embodiments±1%, in some embodiments±0.5%, and in some embodiments±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The methods described herein generally involve evaluating an aspirated sample, e.g., a biological sample, for the presence of extracellular bacteria and/or infection. In some embodiments, the biological sample is a tissue or fluid. In some embodiments, the biological sample is an aspirated tissue or fluid. In embodiments, the biological sample is obtained from a lesion in a subject. In some embodiments, the biological sample is obtained via fine needle aspiration (FNA).

Embodiments described herein utilize sterilized reagents in single-use vials. By using sterilized reagents in single-use vials, one of the largest contributors to artifactual bacterial contamination in conventional detection is mitigated. As used herein, “artifactual bacteria” refers to bacteria that appear in a sample as a result of contamination or external introduction rather than being present in the original source (i.e., the subject lesion) being studied. This may occur during the collection, handling, or processing of samples, leading to incorrect results in microbiological or clinical analyses. Artifactual bacteria can arise from various sources, including environmental contaminants, improper sterilization of equipment, or human handling errors. With the methods described herein, limited artifactual bacteria should be present in the sample.

It will be appreciated that imaging the sample mixture according to the presently disclosed methods enables the cells in the sample to maintain their spherical morphology during imaging, unlike conventional FNA glass slide analyses, which cause the cells to flatten. Extracellular bacteria can be identified in a conventional glass slide preparation or in a fluid sample mixture, such as described herein. By imaging the cells in the sample mixture, detection of extracellular bacteria present in the sample mixture is possible at effectively higher concentrations than on a traditional glass preparation, thereby allowing earlier detection of an infection, more sensitive testing, and removes subjective analysis of the sample. For example, and without being bound by theory, by concentrating more bacteria in the field of vision with the sample mixture, a more uniform distribution is presented. Pairing this with sterility that reduces artifactual bacteria described herein provides a sensitive detection mechanism for bacteria. In addition, it takes time for immune cells to consume bacteria. In contrast, traditional detection mechanisms, while specific for active infections are much less sensitive than the methods disclosed herein.

As used herein, “subject” refers to a veterinary or human subject. In some embodiments, the subject is a mammalian or avian subject. Non-limiting examples of veterinary subjects include equine subjects, ruminant subjects, poultry subjects, swine subjects, canine subjects, feline subjects, and the like. In some embodiments, the subject has a lesion, such as a mass, sore, ulcer, cyst, abscess, abnormal growth, or the like. In some embodiments, the subject may be showing signs of inflammation or infection in a body fluid, such as urine, semen, blood, saliva, sweat, tears, bile, mucus, gastric juices, lymph, breast milk, sputum, serum, colostrum, and/or body cavity fluids, such as peritoneal fluid, pleural fluid, pericardial fluid, synovial fluid, cerebrospinal fluid, amniotic fluid, seminal fluid, aqueous humor and/or vitreous humor. In embodiments, the lesion is a fluid-filled lesion, a solid lesion, and/or an ulcerative lesion.

The term “biological sample,” as used herein, refers to a biological sample obtained from a subject. In embodiments, the biological sample is obtained from the lesion of the subject. In some embodiments, the biological sample comprises cells. It will be appreciated that the amount of liquid present in the biological sample may vary depending on the size of the lesion, the location of the lesion, and the type of tissue or fluid being sampled. For example, and without being bound by theory, the volume of the biological sample extracted from the lesion may range from about 100 microliters (μL) to about 1 milliliter (mL). In some embodiments, the biological sample includes mostly cells and/or other solid matter and does not have an appreciable liquid volume. In some embodiments, the biological sample is extracted from the lesion using a needle. In embodiments, the biological sample is collected in a sample receptacle. In some embodiments, the biological sample is extracted via needle biopsy, such as via fine needle aspiration.

It will be appreciated that, if the surface of the skin is contaminated, bacteria from the surface will contact the needle and may adhere to the outer surface of the needle. In some embodiments, the lesion is sterilized prior to collection of the biological sample. In embodiments, the lesion is sterilized via transient application of a disinfectant suitable for cleansing the skin, such as, but non-limited to, alcohol based solutions, chlorhexidine, iodine-based solutions, hydrogen peroxide, quaternary ammonium compounds, octenidine, and the like.

In some embodiments, the biological sample is diluted with a sterilized diluent to form a sample mixture. In some embodiments, the biological sample is expelled from the sample receptacle into a vial holding the sterilized diluent, for example via a needle. In some embodiments, after the biological sample is expelled, the sample mixture is drawn into the sample receptacle via the needle. In embodiments, the sample mixture is expelled from the sample receptacle via the needle. In some embodiments, the sample mixture is expelled into the single-use vial. In some embodiments, the sample mixture is expelled into a sample cartridge, described in greater detail herein. In embodiments, the needle is sterilized prior to expulsion of the biological sample. Any suitable method of sterilization is contemplated and possible, including chemical sterilization, ultraviolet sterilization, heat sterilization, and the like.

In some embodiments, the sterilized diluent is added to the sample receptacle holding the biological sample. For example, and without being bound by theory, the sterilized diluent may be drawn through a needle into the sample receptacle. In some embodiments, the sterilized diluent may be poured, titrated, or otherwise added to the sample receptacle. In some embodiments, the biological sample and the sterilized diluent are combined in an interior volume of a sample cartridge, described in greater detail herein.

The sterilized diluent is generally a liquid or a solution used to dilute or reduce the concentration of the liquid biological sample. The sterilized diluent may be used to create a more suitable environment for imaging processes, ensuring that the sample is at the right concentration and conditions for accurate analysis. The sterilized diluent may be a water-based solution, a phosphate-buffered saline (PBS) based solution, a tris-buffered saline (TBS) based solution, or a salt-based solution. In some embodiments, the sterilized diluent is a phosphate-buffered saline solution. The sterilized diluent may provide a stable pH level and appropriate ionic strength for the liquid biological sample.

In embodiments, the sterilized diluent may be added to the biological sample in an amount ranging from about 50 microliters to about 1 milliliter, including about 50 microliters, about 60 microliters, about 70 microliters, about 80 microliters, about 90 microliters, about 100 microliters, about 150 microliters, about 200 microliters, about 250 microliters, about 300 microliters, about 350 microliters, about 400 microliters, about 450 microliters, about 500 microliters, about 550 microliters, about 800 microliters, about 850 microliters, about 900 microliters, about 950 microliters, and about 1 milliliter, including any range having endpoints defined by any two of the aforementioned values.

In some embodiments, the sterilized diluent may include a buffer to maintain a desired pH level. In some embodiments, the desired pH level is from about 6.5 to about 8.5, including about 6.6, 6.7, 6.8, 6.9, 7.0, 7.1, 7.2, 7.3, 7.4, 7.5, 7.6, 7.7, 7.8, 7.9, 8.0, 8.1, 8.2. 8.3, and 8.4, including any range having endpoints defined by any two of the aforementioned values. In some embodiments, the sterilized diluent has a pH of about 7.7 to about 8.1. In some embodiments, the sterilized diluent has a pH of about 7.4 to about 7.8.

Any suitable buffer is contemplated and possible. Exemplary buffers include, but are not limited to, phosphate buffers, tris buffers, HEPES buffers, MES buffers, PIPES buffers, MOPS buffers, bicarbonate buffers, glycine buffers, borate buffers, combinations thereof, and the like, In some embodiments, the sterilized diluent includes a phosphate buffer.

In some embodiments, the sterilized diluent may include one or more osmotic agents to maintain a desired osmolarity or osmolality. In some embodiments, the osmotic agents are present in the sterilized diluent at a concentration of about 100 to about 400 mOsm, including about 100, 120, 130, 140, 150, 160, 170, 180, 190, 200, 210, 220, 230, 240, 250, 260, 270, 280, 290, 300, 310, 320, 325, 330, 340, 350, 360, 370, 375, 380, and 390, including any range having endpoints defined by any two of the aforementioned values. In some embodiments, the osmotic agent is present at a concentration of about 330 mOsm to about 350 mOsm. In some embodiments, the osmotic agent is present at a concentration of about 335 mOsm to about 345 mOsm. In some embodiments, the osmotic agent is present at a concentration of about 100 mOsm to about 150 mOsm. In some embodiments, the osmotic agent is present at a concentration of about 110 mOsm to about 140 mOsm. In some embodiments, the osmotic agent is present at a concentration of about 110 mOsm to about 120 mOsm.

Any suitable osmotic agent is contemplated and possible. Exemplary, non-limiting osmotic agents include sodium chloride, calcium chloride, sodium sulfate, sodium bicarbonate, ammonium chloride, sodium acetate, sodium citrate, zinc sulfate. mannitol, glycerol, dextrose, sorbitol, potassium chloride, magnesium sulfate, urea, combinations thereof and the like. In some embodiments, the osmotic agent is sodium chloride.

In some embodiments, the sterilized diluent comprises an imaging agent. For example, in some embodiments, the imaging agent labels a nucleic acid, such as DNA and/or RNA. In some embodiments, the sterilized diluent comprises a plurality of imaging agents. In some embodiments, DNA and RNA are marked by different imaging agents. Exemplary, non-limiting imaging agents include fluorescent dyes, fluorescent stains, fluorescent probes, radioactive labels, quantum dots, labeled antibodies, gram staining, or molecular beacons.

In some embodiments, the imaging agent is a florescent dye or a fluorescent stain. As used herein, a “florescent dye” refers to a molecule that absorbs light at one wavelength (the excitation wavelength) and emits light at a longer wavelength (the emission wavelength) for detection in various biological assays. As used herein, a “fluorescent stain” is a type of fluorescent dye that is specifically chosen for its ability to bind to a particular cellular component (e.g., DNA, RNA, proteins) and produce a fluorescent signal that can be visualized under a microscope or detected using other imaging equipment.

Non-limiting examples of fluorescent dyes and/or fluorescent stains that label DNA include 4′,6-diamidino-2-phenylindole (DAPI), hoescht stains, Syto-13, SYBR green I, ethidium bromide, YO-PRO-1, YOYO-1, DiYO-1, TOTO-1, TOTO-3, DiTO-1, acridine orange, propidium iodide, 7-aminoactinomycin D (7-AAD), PicoGreen, EvaGreen, OliGreen, acridine homodimer, 6-Chlor-2-methoxy-9-acridinamin (ACMA), Texas Red, dihydrocthidium, ethidium homodimers, hydroxystilbamidine, LDS 751, Alexa Fluor, Janelia Fluor (JF) dyes, phthalocyanines, squaraine dyes, perylene diimide (PDI), combinations thereof, and the like, though any suitable fluorescent dye and/or fluorescent stain is contemplated and possible.

Non-limiting examples of fluorescent dyes and/or fluorescent stains that label RNA include RiboGreen, SYBR gold, SYBR green II, acridine orange, fluorescein, rhodomine, Oregon green, TAMRA, Texas Red, Alexa Fluor, boron dipyrromethene (BODIPY), HEX, ROX, SYPRO, fluorescein, tetramethylrhodamine, propidium iodide, ethidium bromide, LDS 751, hydroxystilbamidine, TOTO-1, YOYO-1, YO-PRO-1, Janelia Fluor (JF) dyes, phthalocyanines, squaraine dyes, perylene diimide (PDI), combinations thereof, and the like, though any suitable fluorescent dye and/or fluorescent stain is contemplated and possible.

In embodiments, the imaging agent is present at a concentration from about 5 mg/L to about 75 mg/L, including about 10 mg/L, 15 mg/L, 20 mg/L, 25 mg/L, 30 mg/L, 35 mg/L, 40 mg/L, 45 mg/L, 50 mg/L, 55 mg/L, 60 mg/L, 65 mg/L, and 70 mg/L, including any range having endpoints defined by any two of the aforementioned values. In some embodiments, the imaging agent is present at a concentration from about 0.05 mL/L to about 1 mL/L, including about 0.1 mL/L, 0.2 mL/L, 0.25 mL/L, 0.3 mL/L, 0.4 ml/L, 0.5 mL/L, 0.6 mL/L, 0.7 mL/L, 0.75 mL/L, 0.8 mL/L, and 0.9 mL/L, including any range having endpoints defined by any two of the aforementioned values.

In some embodiments, the sterilized diluent has a plurality of imaging agents. In some embodiments, the imaging agents are hoescht and acridine orange. In some embodiments, the hoescht is present at a concentration from about 10 mg/L to about 50 mg/L. In some embodiments, the acridine orange is present at a concentration from about 0.1 mL/L to about 0.3 mL/L.

In some embodiments, the sterilized diluent includes sucrose. In some embodiments, the sucrose is present at a concentration from about 25 g/L to about 100 g/L, including about 30 g/L, about 40 g/L, about 50 g/L, about 60 g/L, about 70 g/L, about 75 g/L, about 80 g/L, about 85 g/L, about 90 g/L, and about 95 g/L, including any range having endpoints defined by any two of the aforementioned values.

In some embodiments, the sterilized diluent includes serum albumin, such as bovine serum albumin. In some embodiments, the serum albumin is present at a concentration from about 1.0 g/L to about 5 g/L, including about 1.5 g/L, about 2.0 g/L, about 2.5 g/L, about 3.0 g/L, about 3.5 g/L, about 4.0 g/L, and about 4.5 g/L, including any range having endpoints defined by any two of the aforementioned values. In some embodiments, the serum albumin is present at a concentration from about 1.5 g/L to about 3.5 g/L.

In some embodiments, the sterilized diluent includes a plurality of polyacrylate beads. In some embodiments, the polyacrylate beads have an average diameter of about 4 μm to about 6 μm. In some embodiments, the polyacrylate beads are present at a concentration of about 5,000 to about 10,000 beads per μL, including about 6,000, about 7,000, about 8,000, and about 9,000, including any range having endpoints defined by any two of the aforementioned values.

In some embodiments, the sterilized diluent is provided in a single-use vial. As used herein the term “single-use” is used to indicate that the component (e.g., the vial) is intended to be used once and discarded after use. It will be appreciated that a single-use vial reduces the risk of contamination and minimizes the potential for repeated exposure to contaminants inherent in multi-use containers. By using sterilized reagents in single-use vials, one of the largest contributors to artifactual bacterial contamination in conventional detection is mitigated. Artifactual bacteria can arise from various sources, including environmental contaminants, improper sterilization of equipment, or human handling errors. Detecting and accounting for artifactual bacteria is crucial to ensure the accuracy and reliability of scientific and medical findings.

Furthermore, single-use vials provide convenience and efficiency by simplifying the preparation process, eliminating the need for handling and measuring reagents from larger containers and thereby reducing the likelihood of handling errors.

In embodiments, the sample mixture is imaged using an imaging system, such as a microscopy system. It will be appreciated that any suitable imaging system may be used to image the sample mixture.

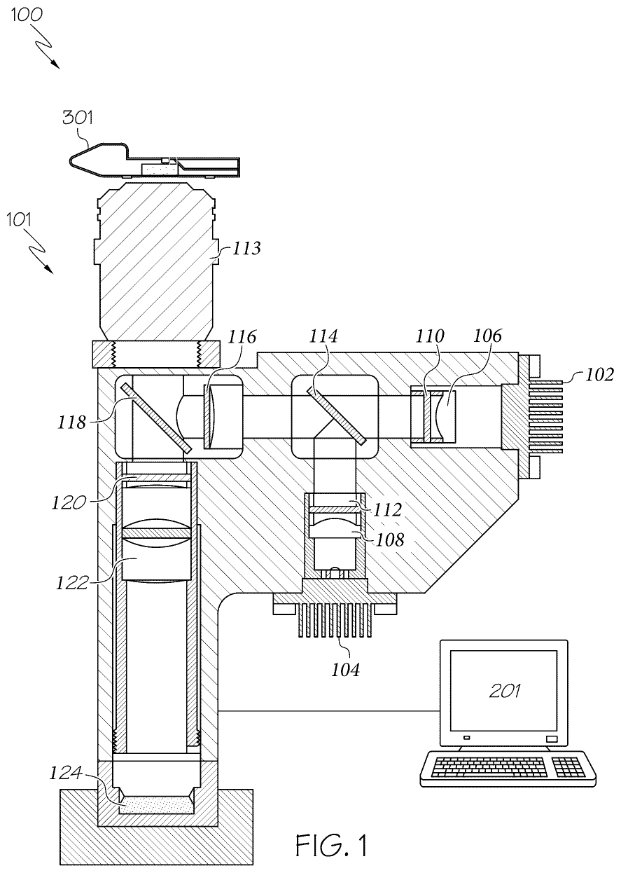

Turning now to the figures, and specifically FIG. 1, an exemplary biological imaging system 100 is depicted. The system 100 generally includes a microscopy device 101 and a controller 201, which are communicatively coupled.

The microscopy device 101 may be any suitable microscopy device. Exemplary, non-limiting microscopy devices include an optical microscopy device, a light microscopy device, an electron microscopy device, a confocal microscopy device, a multiphoton microscopy device, and/or a fluorescence microscopy device. In some embodiments, the microscopy device 101 is a digital microscope. In some embodiments, the microscopy device 101 includes one or more of light sources 102, 104, one or more lenses 106, 108, one or more excitation filters 110, 112, an excitation dichroic 114, one or more field lenses 116, a mirror 118; an objective lens 113, a filter 120, a tube lens 122; one or more imaging sensors 124, one or more specimen stages; and any other components and/or parts suitable for the operation of the microscopy device 101 or to enhance the functionality of the microscopy device 101.

In embodiments, the microscopy device 101 includes the one or more light sources 102, 104 which can vary in number. Non-limiting examples of suitable light sources include continuous spectrum light sources, multichromatic light sources, single-wavelength light sources, and fluorescence light sources of various wavelengths. The pathways for these light sources intersect with the objective optical pathway, allowing optical signals to be modified by optical devices such as mirrors or lenses positioned within specific regions. These optical devices help redirect or reflect the light signals to facilitate imaging.

In embodiments, the imaging sensor 124, is a camera, and may be coupled to the microscopy device 101, and a light source may be positioned opposite the imaging sensor 124 (e.g., above a cartridge 301 as depicted in FIG. 1), optically coupled to the imaging sensor 124 via the objective optical pathway. The term “optically coupled,” as used herein with reference to one or more first optical devices (for example, the objective lens 113) and one or more second optical devices (for example, an imaging sensor 124) means the optical devices are positioned, oriented, and/or designed such that optical signals (for example, for example, visible light, ultraviolet light, blue light, white light, fluorescence light, infrared light, other electromagnetic waves, and/or any combination thereof) may propagate between the one or more first optical devices and the one or more second optical devices. Further, devices and/or components may be said to be “optically coupled” when each device and/or component is within the same optical path of an optical signal transmitted between the devices and/or components. Accordingly, the term “optical path” when used herein with respect to an optical signal is the path of the optical signal transmitted by, between, and/or through one or more devices and/or components.

Accordingly, by being optically coupled, the imaging sensor 124 may capture images of the sample cartridge 301 positioned opposite the objective lens 113 from the imaging sensor 124 by detecting optical signals propagating through the sample cartridge 301 and the objective lens 113.

The sample cartridge 301 may be, without limitations, a cartridge for bio-microscopy imaging. In embodiments, the sample cartridge 301 may be a container having a transparent bottom and a top opposite the transparent bottom. The sample cartridge 301 may have several depths. In some embodiments, the sample cartridge 301 is placed adjacent to the microscopy device 101 to be imaged. As used herein “adjacent” refers to objects, components, or features that are located next to each other in a manner that allows for direct or indirect interaction, contact, or influence. It will be appreciated that this term encompasses both items that are directly touching and those separated by an intermediate element, provided they are sufficiently close to affect one another's function or operation.

In embodiments, the microscopy device 101 may further include and/or be coupled to a stage, upon which sample cartridge 301 is positioned. In embodiments, a light source, such as the light sources 102, 104, may be positioned opposite an imaging sensor 124 such that, optical signals generated by the light source may propagate through the sample cartridge 301 and the objective lens 113 and to the imaging sensor 124.

In some embodiments, the microscopy device 101 may switch between, a brightfield mode, a darkfield mode, and/or a fluorescent mode. In the brightfield mode, a condenser lens (not depicted) may focus and direct the light onto the sample mixture, creating a bright and uniform background. The objective lens 113 may collect the transmitted light from the sample and direct it to the imaging sensor 124.

In the dark field mode, the condenser lens (not depicted) may scatter or refract light to reach the objective lens 113, and the objective lens 113 may collect this scattered light and form an image of the sample mixture where the detected bacteria appear bright against a dark background.

In the fluorescent mode, one or more excitation filters 110, 112 of different specific wavelengths may be used to select the excitation light that matches the absorption spectrum of the fluorescent dye or stain in the sample mixture. An imaging dichroic mirror 118 may act as a beam splitter and reflect the excitation light towards the sample cartridge 301 while allowing emitted fluorescence to pass through the objective lens 113 to the imaging sensor 124. The objective lens 113 may collect the emitted fluorescence from the extracellular bacteria that is stained with the fluorescent dye or stain.

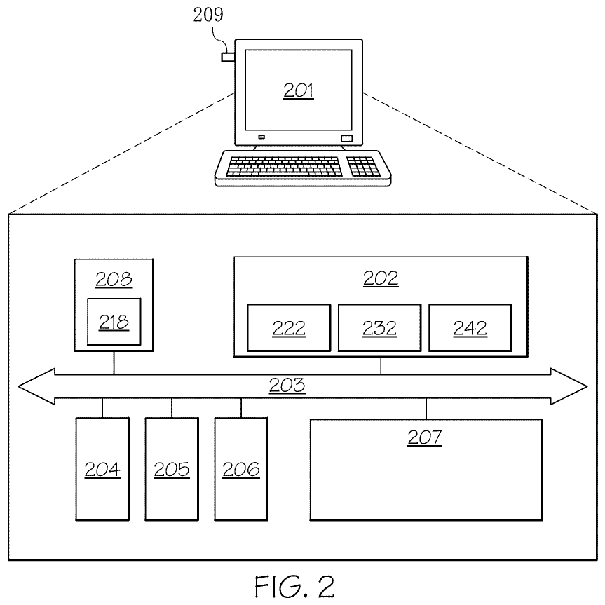

The biological imaging system 100 includes the controller 201, as shown in FIG. 2. The controller 201 includes one or more connections 209. The one or more connections 209 connect components of the microscopy device 101 to the controller 201 and allow signal transmission between the components of the biological imaging system 100. The connections 209 may be formed from any medium that is capable of transmitting a signal such as, for example, conductive wires, conductive traces, optical waveguides, or the like. In some embodiments, the connections 209 may facilitate the transmission of wireless signals, such as WiFi, Bluetooth®, Near Field Communication (NFC) and the like. Moreover, the connections 209 may be formed from a combination of mediums capable of transmitting signals. In one embodiment, the connections 209 may include a combination of conductive traces, conductive wires, connectors, and buses that cooperate to permit the transmission of electrical data signals to components such as processors, memories, sensors, input devices, output devices, and communication devices. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium. The controller 201 may receive inputs from the components and provide outputs to the components of the microscopy device 101.

Still referring to FIG. 2, exemplary, non-limiting components of the controller 201 are depicted. The controller 201 may include various components, such as a memory component 202, a processor 204, an input/output interface 205, a network interface hardware 206, a data storage component 207, a display 208 including a user interface 218, and a local interface 203. The controller 201 may include one or more displays 208 with one or more user interfaces 218. The controller 201 may include one or more modules, such as a biological sample module 222 to detect extracellular bacteria in the sample mixture, a machine learning module 232, and a focusing module 242 to conduct real-time sequence focusing at various depths of the sample mixture as described herein.

The controller 201 may be any device or combination of components comprising a processor 204 and a memory component 202, such as a non-transitory computer readable memory. The processor 204 may be any device capable of executing a machine-readable instruction set stored in the non-transitory computer readable memory. Accordingly, the processor 204 may be an electric controller, an integrated circuit, a microchip, a computer, or any other computing device. The processor 204 may include any processing component(s) configured to receive and execute programming instructions (such as from the data storage component 207 and/or the memory component 202). The instructions may be in the form of a machine-readable instruction set stored in the data storage component 207 and/or the memory component 202. The processor 204 is communicatively coupled to the other components of the controller 201 by the local interface 203. Accordingly, the local interface 203 may communicatively couple any number of processors 204 with one another, and allow the components coupled to the local interface 203 to operate in a distributed computing environment. The local interface 203 may be implemented as a bus or other interface to facilitate communication among the components of the controller 201. While the embodiment depicted in FIG. 2 includes a single processor, other embodiments may include more than one processor.

The memory component 202 (e.g., a non-transitory computer-readable memory component) may include RAM, ROM, flash memories, hard drives, or any non-transitory memory device capable of storing machine-readable instructions such that the machine-readable instructions can be accessed and executed by the processor 204. The machine-readable instruction set may include logic or algorithm(s) written in any programming language of any generation (e.g., 1GL, 2GL, 3GL, 4GL, or 5GL) such as, for example, machine language that may be directly executed by the processor 204, or assembly language, object-oriented programming (OOP), scripting languages, microcode, etc., that may be compiled or assembled into machine readable instructions and stored in the memory component 202. Alternatively, the machine-readable instruction set may be written in a hardware description language (HDL), such as logic implemented via either a field-programmable gate array (FPGA) configuration or an application-specific integrated circuit (ASIC), or their equivalents. Accordingly, the functionality described herein may be implemented in any conventional computer programming language, as pre-programmed hardware elements, or as a combination of hardware and software components. For example, the memory component 202 may be a machine-readable memory (which may also be referred to as a non-transitory processor-readable memory or medium) that stores instructions that, when executed by the processor 204, causes the processor 204 to perform a method or control scheme as described herein. While the embodiment depicted in FIG. 2 includes a single non-transitory computer-readable memory component, other embodiments may include more than one memory module. The memory component 202 may be used to store the one or more modules. The one or more modules during operating may be in the form of operating systems, application program modules, and other program modules. Such program modules may include, but are not limited to, routines, subroutines, programs, objects, components, and data structures for performing specific tasks or executing specific abstract data types according to the present disclosure as will be described below. For example, the program module may include the biological sample module 222 to detect extracellular bacteria in the sample mixture, a machine learning module 232, and a focusing module 242 to conduct real-time sequence focusing at various depths of the sample mixture as described herein.

The input/output interface 205 may include a monitor, keyboard, mouse, printer, camera, microphone, speaker, and/or other device for receiving, sending, and/or presenting data. The network interface hardware 206 may include any wired or wireless networking hardware, such as a modem, LAN port, Wi-Fi card, WiMax card, mobile communications hardware, and/or other hardware for communicating with other networks and/or devices. The data storage component 207 may store the one or more modules. The input/output interface 205 and the network interface hardware 206 allow a user to send input to the controller 201 of the biological imaging system 100 to control and manipulate the components of the biological imaging system 100, such as the one or more of light sources 102, 104, one or more lenses 106, 108, one or more excitation filters 110, 112, the excitation dichroic 114, the one or more field lenses 116, the mirror 118; the objective lens 113, the filter 120, the tube lens 122, and/or the imaging sensor 124, and receive output from the controller 201.

In operation, the imaging sensor 124 may capture image data and communicates the image data to the processor 204. The image data may be received by the processor 204, which may process the image data using one or more image processing algorithms. Any known or yet-to-be developed video and image processing algorithms may be applied to the image data to identify an item or situation. Example video and image processing algorithms include, but are not limited to, kernel-based tracking (such as, for example, mean-shift tracking) and contour processing algorithms. In general, video and image processing algorithms may detect objects and movement from sequential or individual frames of image data. One or more object recognition algorithms may be applied to the image data to extract objects and determine their relative locations to each other. Any known or yet-to-be-developed object recognition algorithms may be used to extract the objects or even optical characters and images from the image data. Example object recognition algorithms include, but are not limited to, scale-invariant feature transform (“SIFT”), speeded up robust features (“SURF”), and edge-detection algorithms.

In embodiments, the image processing algorithm(s) may generate data corresponding to certain attributes of the sample mixture, including but not limited to bacterial cell count.

In some embodiments, the biological sample module 222 may include an algorithm to identify and differentiate the bacteria from other components of the sample mixture. The algorithm may adopt one or more offsets in differentiating the bacteria based on the size, the fluorescent wavelength, or the surface morphology between the bacteria, debris, and/or cells or cellular material.

For example, and without being bound by theory, the size of the extracellular bacteria may be from about 0.1 micrometers to about 8 micrometers. The surface morphology of the bacteria may be cocci, rod-shaped, bacilli, spirilla, spirochete, vibrio, and/or coccobacillus. Cells in the sample mixture may be from about 10 micrometers to about 100 micrometers. The surface morphology of the cells may be spherical, cuboidal, columnar, squamous, stellate, spindle, discoid, and/or amorphous.

In some embodiments, imaging the sample mixture comprises brightfield microscopy, fluorescent microscopy, or a combination thereof. In embodiments, the biological imaging system 100 quantifies the amount of extracellular bacteria in the sample mixture. In some embodiments, the amount of bacteria in the sample mixture is determined by measuring the signal from the imaging agent.

For example, and without being bound by theory, when the imaging agent is a fluorescent dye or stain, the system 100 uses specific wavelengths of light to excite the fluorescent dye or stain, causing them to emit light at different wavelengths. It will be appreciated that the specific wavelengths of light will differ based on the particular fluorescent dye or stain used. In some embodiments, the emitted light from the sample mixture is captured by the imaging sensor 124, as described in greater detail herein. In some embodiments, the imaging sensor 124 communicates the image data to the processor 204 which processes the image using one or more image processing algorithms. In some embodiments, the image processing algorithm distinguishes between background fluorescence and the specific fluorescence emitted by the stained bacteria.

In some embodiments, without being bound by theory, the imaging agent binds to the extracellular bacteria. The sample mixture is imaged using light which illuminates the sample mixture. The imaging sensor 124 captures this image data, where the bacteria appear as distinct shapes against the background. In some embodiments, the imaging sensor 124 communicates the image data to the processor 204 which processes the image using one or more image processing algorithms. In some embodiments, the image processing algorithm distinguishes bacteria from the background based on contrast and morphological features.

In embodiments, the system 100 provides an output. In some embodiments, this output provides an indication of a likelihood of infection. In some embodiments, the output is provided on a user interface, a text report, or stored data. In some embodiments, the output provides a count of bacterial cells, a concentration of bacteria, and/or a presence or absence of infection.

In embodiments, when extracellular bacteria are present in the sample mixture, extracellular bacteria is present in the liquid biological sample. In some embodiments the presence of extracellular bacteria in the liquid biological sample is an indicator of infection. In some embodiments, the presence of extracellular bacteria above a threshold value is indicative of an infection.

In some embodiments, the biological imaging system 100 may define a threshold value to determine whether the bacteria in the sample mixture is indicative of an infection. The threshold serves as a cutoff point, above which an infection is considered likely and below which it is considered unlikely. For example, if the number of bacteria present within the sample mixture is above the threshold, the liquid biological sample is determined to have extracellular bacteria indicative of infection. In some embodiments, the threshold value is a configurable threshold. The configurable threshold may be predetermined based on one or more of the location of the lesion, the actual or theoretical bacterial load of the sampling location, the volume of the liquid biological sample, the size of the needle used to collect the liquid biological sample, or combinations thereof.

In some embodiments, the threshold value is determined based on an expected amount of artifactual bacteria in the sample mixture. For example, and without being bound by theory, in some embodiments where the liquid biological sample is obtained from a lesion in a sampling location with a high contaminant bacteria load (e.g., oral cavity, perineal area; vagina, nasopharynx, gastrointestinal tract, urethra, etc.), the threshold value may be configured to account for the higher baseline presence of bacteria in these areas. In some embodiments, for example when the lesion site is sterilized prior to collection of the liquid biological sample and/or the needle is sterilized after the collection of the liquid biological sample but prior to expulsion, the threshold value may be configured to account for the lower bacterial contamination.

In some embodiments, the configurable threshold may be input or set by an operator. In some embodiments, the operator may input parameters or characteristics of the liquid biological sample and/or sampling site into the biological sampling module. In such embodiments, the system may calculate the threshold value. In some embodiments, the threshold value may evolve as more data are collected across various parameters using a machine learning module 232 associated with the biological sample module 222.

In some embodiments, the configurable threshold is less than about 500 bacteria per microliter, including about 10 bacteria per microliter, about 15 bacteria per microliter, about 20 bacteria per microliter, about 25 bacteria per microliter, about 30 bacteria per microliter, about 35 bacteria per microliter, about 40 bacteria per microliter, about 45 bacteria per microliter, about 50 bacteria per microliter, about 55 bacteria per microliter, about 60 bacteria per microliter, about 65 bacteria per microliter, about 70 bacteria per microliter, about 75 bacteria per microliter, about 80 bacteria per microliter, about 85 bacteria per microliter, about 90 bacteria per microliter, about 95 bacteria per microliter, about 100 bacteria per microliter, about 110 bacteria per microliter, about 120 bacteria per microliter, about 125 bacteria per microliter, about 130 bacteria per microliter, about 140 bacteria per microliter, about 150 bacteria per microliter, about 175 bacteria per microliter, about 180 bacteria per microliter, about 190 bacteria per microliter, about 200 bacteria per microliter, about 210 bacteria per microliter, about 220 bacteria per microliter, about 225 bacteria per microliter, about 250 bacteria per microliter, about 275 bacteria per microliter, about 300 bacteria per microliter, about 325 bacteria per microliter, about 350 bacteria per microliter, about 375 bacteria per microliter, about 400 bacteria per microliter, about 425 bacteria per microliter, about 450 bacteria per microliter, about 475 bacteria per microliter, and about 500 bacteria per microliter, including any range having endpoints defined by any two of the aforementioned values.

In some embodiments, for example, when the bacteria present in the sample mixture is not above the configurable threshold, at least a portion of the bacteria present is determined to be from contamination. For example, and without being bound by theory, in some embodiments, the presence of bacteria at low concentrations may be considered within the normal range and is typically managed by neutrophils without the need for external intervention, such as antibiotic treatment. In some embodiments, this low-level threshold encompasses bacteria originating from the lesion that fall below the detection threshold, artifactual bacteria below the threshold, or a combination of both. In some embodiments, the system may detect a “gray-zone” near the threshold where the system may detect bacterial presence and recommend further evaluation, such as incorporating clinical signs or conducting additional bacterial-specific testing if deemed necessary. In some embodiments, contaminant bacteria, such as those from the oral cavity, often exhibit distinct size and morphology compared to bacteria that would typically infect a lesion, allowing for their differentiation from bacteria indicative of infection. In some embodiments, described in greater detail herein, the sample mixture is evaluated for the presence of neutrophils or other immune cells to further confirm the indication of infection.

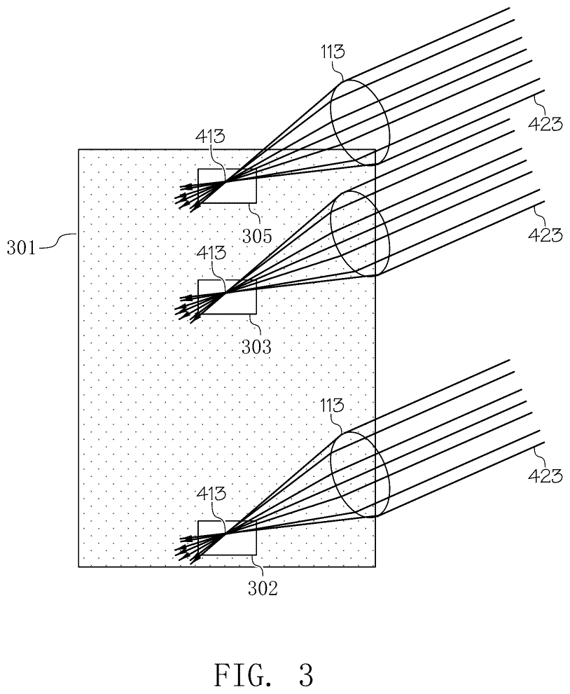

Referring to FIG. 3, with reference to the system 100 illustrated in FIG. 1, various imaging modes are depicted. After the sample cartridge 301 is placed in the well or chamber of the microscopy device 101, the system 100 may focus, using the focusing module 242 (FIG. 2) at different depths of the solution to detect bacteria or other constituents of the sample mixture. In some embodiments, the focusing module 242 positions the focal point 413 of the microscopy device 101 by manipulating the objective lens 113 to refract incoming light 423 and achieve focus at one or more specified depths of interest. In embodiments, the specified depth is above a plurality of settled constituents (such as the polyacrylate beads described in greater detail herein) in the sample mixture, described in greater detail herein.

In some embodiments, the focal point 413 may be controlled to shift within a region of interest, such as near the surface 305 of the sample cartridge 301, expected to contain extracellular bacteria, as described herein, near a lowermost portion 302 of the sample cartridge 301, expected to contain settled constituents, such as the polyacrylate beads, cellular material and other debris, and/or a region 303 in between the surface 305 and the lowermost portion 302.

The system 100 may capture one or more images of the sample mixture. The system 100 can detect extracellular bacteria and/or other constituents in the sample mixture at various depths of interest. In some embodiments, the system 100 utilizes the biological sample module 222 to analyze the images and determine the presence or absence of extracellular bacteria above the threshold value. This analysis informs further actions for the microscopy device 101, and it also communicates messages to the user through the user interface 218.

It has been observed that bacteria in liquid samples exhibit behaviors that differentiate them from non-bacterial debris and cellular material. Bacteria tend to auto-arrange into a lattice-like grid pattern uniformly spaced throughout the fluid sample and remain suspended in the fluid, unlike other components that settle at the bottom. This behavior is consistent and can be used to reliably identify and measure bacterial concentration in the sample mixture. The auto-arrangement is driven by forces like zeta potential, counteracting gravitational settling and maintaining bacteria in a stable structure. Bacteria also demonstrate an aversion to surfaces, creating a “no-bacteria zone” near container walls. They exhibit uniform sizing and distribution, unlike debris which clusters and has irregular shapes. After a period of time, bacteria separate from settled elements and remain in suspension.

These characteristics—auto-arrangement and the no-bacteria zone—are indicators for distinguishing bacteria from non-bacterial particles, even differentiating bacterial forms. These phenomena also enable differentiation of the cellular material from the bacteria when both give a signal from the imaging agent.

In some embodiments, the sample mixture is imaged at a z-axis depth of the interior volume of the sample cartridge 301. In some embodiments, the z-axis is positioned above the plurality of settled constituents. It will be appreciated that the “z-axis” refers to the direction perpendicular to the imaging plane. It will be further appreciated that imaging the sample mixture in this manner captures clear images of the sample mixture without including the settled constituents in the focal plane.

In embodiments, the sample mixture is imaged after allowing the mixture to equilibrate for a period of time sufficient to allow the plurality of constituents in the sample mixture to settle. In some embodiments, the sample mixture is placed in the sample cartridge and is allowed to equilibrate for a period of time of from about 2 minutes to about 15 minutes, including about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, and about 15 minutes, including any range having endpoints defined by any two of the aforementioned values.

In some embodiments, the settling of the constituents in the sample mixture may be monitored using the system 100. For example, and without being bound by theory, during a focus sequence implemented by focus module 242, the settling of various constituents in the sample mixture may be determined by arranging the focal point 413 in depths around the lowermost portion 302 of the solution and capturing an image of the settled constituents.

In embodiments, the system 100 may first determine whether extracellular bacteria are detected at one or more of the scanned depths of interest. After the detection of the extracellular bacteria at certain depths, the system 100 may further determine whether other constituents, such as cellular material or debris are present in the sample mixture. In embodiments, the system 100 determines whether a leukocyte is present in the sample mixture. In some embodiments, the leukocyte is selected from neutrophils, lymphocytes, monocytes, eosinophils, basophil, or a combination thereof.

For example, and without being bound by theory, in some embodiments, optical sections of the sample cartridge 301 are taken at specific depths or angles. For example, and without being bound by theory, in some embodiments, the optical sections are taken vertically, horizontally, and/or at slanted angles relative to a vertical axis, with focal depths at approximately 50, 100, and 150 microns above the container bottom for horizontal sectioning. The number of extracellular bacteria in each optical section is quantified and averaged, allowing for an approximation of the total bacteria concentration per microliter. In some embodiments, the average spacing between bacteria is measured to estimate concentration.

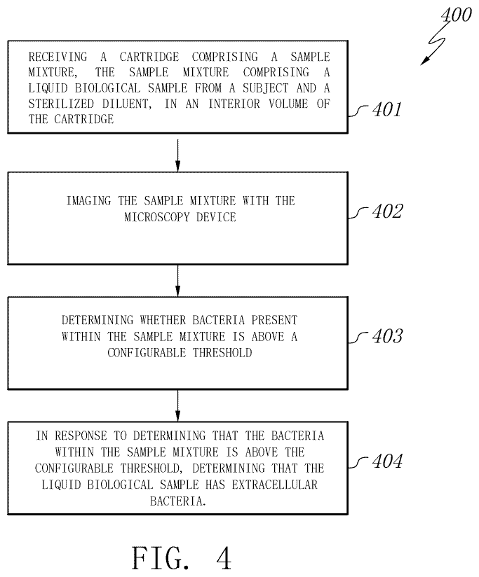

Referring to FIG. 4, a flowchart of an example method 400 of evaluating an aspirated fluid using the biological imaging system 100 is depicted, with reference to FIGS. 1-2 consistent with one or more embodiments as described herein. At step 401, the method includes receiving a sample cartridge 301 comprising a sample mixture, the sample mixture comprising a liquid biological sample from a subject and a sterilized diluent, in an interior volume of the sample cartridge 301. The liquid biological sample and the sterilized diluent may be mixed in the interior volume of the sample cartridge 301. Alternatively, the liquid biological sample and the sterilized diluent may be combined to form the sample mixture and the sample mixture may be added to the interior volume of the sample cartridge 301, as described in greater detail herein.

At step 402, the method includes imaging the sample mixture with the microscopy device 101. Generally, the microscopy device 101 uses the one or more light sources 102, 104, illuminate the sample mixture in the sample cartridge 301. In some embodiments, a condenser focuses the light from the one or more light sources 102, 104. In some embodiments, objective lens 113 collects the light from the sample cartridge 301. The image data is captured by the imaging sensor 124 as described herein. In some embodiments, the imaging sensor 124 converts the captured light into an electronic signal.

In some embodiments, the method 400 includes imaging the mixture at a z-axis depth of the interior volume of the sample cartridge 301 positioned above a plurality of settled constituents. In some embodiments, the method 400 may include arranging a focal point 413 of the microscopy device 101 in one or more depths of interest, such as near the surface 305 of the sample mixture, near a lowermost portion 302 of the sample mixture, or at a region 303 between the two

At step 403, the method includes determining whether bacteria present within the sample mixture is above a configurable threshold. In some embodiments, imaging sensor 124 communicates the image data to the processor 204 which processes the image using one or more image processing algorithms as described herein. In some embodiments, the image processing algorithm(s) may generate data corresponding to certain attributes of the sample mixture, including but not limited to bacterial cell count, a total cell count, a white blood cell count, a platelets count, a neutrophils count, a lymphocytes count, a monocytes count, a eosinophils count, a mean corpuscular volume, a mean platelets volume, a red blood cell distribution width, a platelet distribution width, or any other suitable attributes of the sample mixture, as described herein.

In some embodiments, the method 400 may include an imaging algorithm that adopts one or more offsets, based on the extracellular bacteria and other constituents of the sample mixture in position, size, fluorescent wavelength, and/or surface morphology, to identify the extracellular bacteria and other constituents, such as cells or debris. The size of the extracellular bacteria may be from about 0.1 micrometers to about 8 micrometers. The surface morphology of the bacteria may be cocci, bacilli, spirilla, spirochete, vibrio, and/or coccobacillus. Cells in the sample mixture may be from about 4 micrometers to about 100 micrometers. The surface morphology of the cells may be spherical, cuboidal, columnar, squamous, stellate, spindle, discoid, and/or amorphous.

That is, the processor 204 may compare the image data associated with the sample mixture and generated by the biological imaging system 100 to the configurable threshold to determine if the sample mixture sample is above or below the threshold value. The threshold value, as described herein, may be a bacterial value attributable to artifactual bacteria.

Determining whether bacteria present within the sample mixture is above a configurable threshold at step 402 may further include detecting and quantifying an amount of extracellular bacteria present in the sample mixture. The method 400 may include, in response to determining the bacteria present in the sample mixture is not above the configurable threshold, that at least a portion of the bacteria present is from contamination. For example, and without being bound by theory, in some embodiments, thresholds can be set empirically by applying bacteria at known concentrations to surrogate sample surfaces and aspirating through them into known sterile areas to quantify the potential levels of artifactual bacteria. Similarly, experimentation can be performed with patients who have had the skin sterilized to confirm that there is no bacteria on the surface. The samples can then be evaluated to quantify the bacterial concentrations from the samples. Distributions of known negative, positive, and artifactual bacteria counts from the system can then be compared to determine the desired thresholds.

Still referring to FIG. 4, at step 404, in response to determining that the bacteria within the sample mixture is above the configurable threshold, the method 400 includes determining that the liquid biological sample has extracellular bacteria.

In response to determining the bacteria present in the sample mixture is above the configurable threshold at step 403 and/or in response to determining the liquid biological sample has extracellular bacteria at step 404, the method 400 may include providing an indication of a likelihood of infection. In some embodiments, the indication of a likelihood of infection is provided on a user interface, a text report, or stored data as described herein.

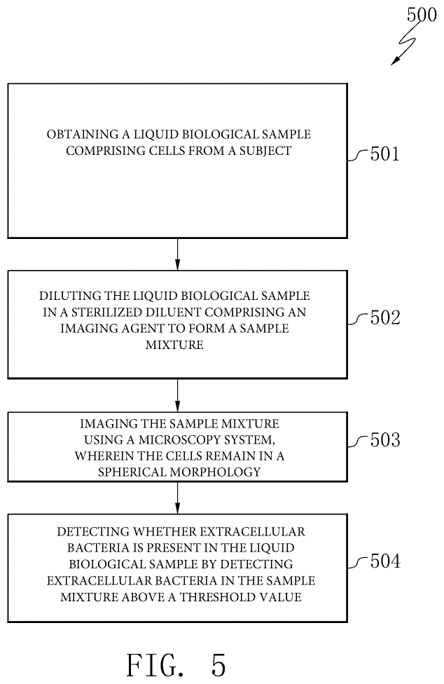

Referring to FIG. 5, a flowchart of an example method 500 of detecting extracellular bacteria is depicted, with reference to FIGS. 1-2. In embodiments, at step 501, the method may include obtaining a liquid biological sample comprising cells from a subject. In embodiments, the cells are extracted from a lesion in the subject as described herein. The lesion may be a fluid filled lesion. In some embodiments, the lesion may be a solid lesion. In some embodiments, the liquid biological sample is extracted via needle biopsy, such as fine needle aspiration.

At step 502, in some embodiments, the liquid biological sample is diluted in a sterilized diluent comprising an imaging agent to form a sample mixture as described herein. In some embodiments, the imaging agent labels DNA. In some embodiments, the imaging agent labels RNA. In some embodiments, the imaging agent is selected from fluorescent dyes, fluorescent stains, fluorescent probes, radioactive labels, quantum dots, labeled antibodies, gram staining, and/or molecular beacons. In some embodiments, the imaging agent is a fluorescent dye or a fluorescent stain. In some embodiments, the liquid biological sample is expelled from a sample receptacle via a needle. In some embodiments, the liquid biological sample is expelled into a vial holding the sterilized diluent. In some embodiments, the vial is a single-use vial. In embodiments, the needle is sterilized prior to expelling the liquid biological sample.

At step 503, the sample mixture is imaged using a microscopy system, such as biological imaging system 100. In some embodiments, the cells remain in a spherical morphology during imaging. In some embodiments, imaging the sample mixture comprises brightfield microscopy, fluorescent microscopy, or a combination thereof. Generally, the biological imaging system 100 may employ the microscopy device 101 to image the sample mixture. The microscopy device 101 may use the one or more light sources 102, 104, to illuminate the sample mixture. In some embodiments, a condenser focuses the light from the one or more light sources 102, 104. In some embodiments, objective lens 113 collects the light from the sample cartridge 301. The image data is captured by the imaging sensor 124 as described herein. In some embodiments, the imaging sensor 124 converts the captured light into an electronic signal. Imaging sensor 124 communicates the image data to the processor 204 which processes the image using one or more image processing algorithms as described herein. In embodiments, the image processing algorithm(s) may generate data corresponding to certain attributes of the sample mixture, including but not limited to bacterial cell count, a total cell count, a white blood cell count, a platelets count, a neutrophils count, a lymphocytes count, a monocytes count, a eosinophils count, a mean corpuscular volume, a mean platelets volume, a red blood cell distribution width, a platelet distribution width, or any other suitable attributes of the sample mixture, as described herein.

At step 504, the method may include detecting whether extracellular bacteria is present in the liquid biological sample by detecting extracellular bacteria in the sample mixture above a threshold value as described herein. In some embodiments, the threshold value is indicative of artifactual bacteria. To detect whether extracellular bacteria is present in the liquid biological sample at step 504, in some embodiments as described herein, imaging sensor 124 communicates the image data to the processor 204 which processes the image using one or more image processing algorithms as described herein. In some embodiments, the image processing algorithm(s) may generate data corresponding to certain attributes of the sample mixture, including but not limited to bacterial cell count, a total cell count, or any other suitable attributes of the sample mixture, as described herein.

In some embodiments, the method 500 may include an imaging algorithm that adopts one or more offsets, based on the extracellular bacteria and other constituents of the sample mixture in position, size, fluorescent wavelength, and/or surface morphology, to identify the extracellular bacteria and other constituents, such as cells or debris. The size of the extracellular bacteria may be from about 0.1 micrometers to about 8 micrometers. The surface morphology of the bacteria may be cocci, bacilli, spirilla, spirochete, vibrio, and/or coccobacillus. Cells in the sample mixture may be from about 4 micrometers to about 100 micrometers. The surface morphology of the cells may be spherical, cuboidal, columnar, squamous, stellate, spindle, discoid, and/or amorphous.

That is, the processor 204 may compare the image data associated with the sample mixture and generated by the biological imaging system 100 to the configurable threshold to determine if the sample mixture sample is above or below the threshold value. The threshold value, as described herein, may be a bacterial value attributable to artifactual bacteria. If the sample mixture is determined to contain more bacteria than the threshold value, the liquid biological sample is determined to contain extracellular bacteria.

In some embodiments, the method 500 may further include detecting a leukocyte in the sample mixture. In embodiments, the leukocyte is selected from neutrophils, lymphocytes, monocytes, eosinophils, basophil, or a combination thereof. As described herein, the method 500 may include an imaging algorithm that adopts one or more offsets, based on the extracellular bacteria and other constituents of the sample mixture in position, size, fluorescent wavelength, and/or surface morphology, to identify the cells and cell types. Cells in the sample mixture may be from about 4 micrometers to about 100 micrometers. The surface morphology of the cells may be spherical, cuboidal, columnar, squamous, stellate, spindle, discoid, and/or amorphous.

As used herein, the term “comprising” means that various other compatible drugs and medicaments, as well as inert ingredients, can be conjointly employed in the pharmaceutical compositions and methods described herein, as long as the defined pharmaceutically active compounds and carriers are used in the manner disclosed. The term “comprising” thus encompasses and includes the more restrictive terms “consisting of” and “consisting essentially of.”

Unless otherwise defined, all technical and scientific terms used herein have the same meaning as commonly understood by one of ordinary skill in the art to which the claimed subject matter belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

It is noted that terms like “preferably,” “commonly,” and “typically” are not utilized herein to limit the scope of the appended claims or to imply that certain features are critical, essential, or even important to the structure or function of the claimed subject matter. Rather, these terms are merely intended to highlight alternative or additional features that may or may not be utilized in a particular embodiment.

### EXAMPLES

The following detailed methodology and materials are set forth to support and illustrate particular aspects and embodiments of the present disclosure, and should not be construed as limiting the scope thereof.

### Example 1: Theoretical Bacterial Contamination

Standard needle gauges for FNA tend to be between 22 gauge and 25 gauge. Table 1 below provides the inner and outer diameter sizes of these gauge needles from Hamilton Laboratory Products:

TABLE 1
Reference sizes for common FNA needle gauges

| Gauge | 22 | 23 | 24 | 25 |
|---|---|---|---|---|
| OD (mm) | 0.718 | 0.642 | 0.566 | 0.515 |
| ID (mm) | 0.413 | 0.337 | 0.311 | 0.26 |

The potential concentration of contaminant bacteria may be calculated to determine the threshold value. Contaminant bacteria may occur on either the needle exterior or the needle interior.

### Needle Exterior

Contaminant bacteria may attach to the outside of the needle shaft when inserted through a contaminated surface, such as a vial topper or a sampling location with a high contaminant bacteria load (e.g., oral cavity, perineal area; vagina, nasopharynx, gastrointestinal tract, urethra, etc.), which may then be transferred into the sample mixture.

To calculate the theoretical contamination of the needle exterior with a high bacterial skin load, such as 10⁹ bacteria per milliliter (10⁶ bacteria per microliter), assuming 100% transfer efficiency, the volume of the surface at a thickness of 1 micron is determined, which is about the diameter of a single bacterium. This creates a monolayer of bacteria on the needle. For a needle piercing 1 cm through the skin, the volume of surface contact ranges from 0.023 microliters for a 22-gauge needle to 0.016 microliters for a 25-gauge needle. At a concentration of 10⁶ bacteria per microliter, this results in approximately 23,000 bacteria on the surface of a 22-gauge needle and approximately 16,000 bacteria on the surface of a 25-gauge needle. Thus, a 1-cm pierce through the skin could theoretically transfer about 23,000 bacteria with a 22-gauge needle and about 16,000 bacteria with a 25-gauge needle. When the needle is then inserted into the sterilized diluent (approximately 250 microliters) to dispense the sample, the bacterial contamination level is 90 bacteria per microliter when a 22 gauge needles is used and 65 bacteria per microliter when a 25-gauge needle is used.

This level of bacterial contamination would not be measurable where a positive concentration of extracellular bacteria from an untreated infection would be on the order of 103 bacteria per microliter or higher.

### Needle Interior

Contaminant bacteria may also attach to the inside of the needle shaft when inserted through a contaminated surface, such as a vial topper or a sampling location with a high contaminant bacteria load (e.g., oral cavity, perineal area; vagina, nasopharynx, gastrointestinal tract, urethra, etc.), which may then be transferred into the sample mixture.

To calculate the theoretical contamination of the needle interior with a high bacterial skin load, such as 10⁹ bacteria per milliliter (10⁶ bacteria per microliter), assuming 100% transfer efficiency, the volume of the surface at a thickness of 1 micron, which is about the diameter of a single bacterium, is determined. When piercing skin contaminated with bacteria, the maximum number of bacteria that could enter the needle during the puncture may be estimated by multiplying the thickness of the bacterial layer by the inner surface area of the needle shaft. This estimation assumes that the needle hole is located at the bottom of the needle, effectively “coring” the skin as it passes through. It will be appreciated that this estimation may overestimate the amount of bacteria, as the hole in the needles is positioned at the bevel tip, thereby limiting coring.

At a concentration of 10⁶ bacteria per microliter, this results in approximately 134 bacteria on the interior surface of a 22-gauge needle and approximately 53 bacteria on the interior surface of a 25-gauge needle. When the needle is then inserted into the sterilized diluent (approximately 250 microliters) to dispense the sample, the bacterial contamination level is less than 1 bacteria per microliter when either a 22 gauge needle or a 25 gauge needle is used.

### Example 2: Confirmation of Infection

Experimental validation will be performed for the described methods. This validation will involve assessing the specificity, linearity, precision, range, detection limit, quantitation limit, robustness, and system suitability. This testing will evaluate both natural samples with bacteria present in the lesion and artificially contaminated samples and will assess the ability of the methods to distinguish between the two conditions.

Purposeful contamination of a needle may be achieved in the laboratory by artificially spiking the surface of a sample with bacteria. Subsequently, various aspiration techniques can be performed to determine the concentration of bacteria transferred to a glass slide preparation (utilizing known sterile stains) and to the fluidic approach described. This testing will quantify the potential bacterial contamination that can be transferred to the sample and will validate the evaluation method.

Purposeful contamination of stains may be conducted to evaluate the potential contaminant load on a glass slide from inoculated stains. This assessment provides reference data on the potential scale of contamination and underscores the benefits of using sterile, single-use reagents.

Positive samples containing intracellular bacteria, identified on glass slide preparations from natural samples, will be used as references for paired samples analyzed in the fluid environment. Samples collected using sterile stains, where extracellular bacteria is identified but intracellular bacteria is not, will also be included. Paired samples will be run in the fluid environment and analyzed accordingly.

Samples may undergo quantitative culture analysis to determine reference bacterial loads. Subsequent analysis of isolated cultures may be conducted to identify bacterial strains, further confirming whether the bacteria are contaminant bacteria or associated with the lesion.

Embodiments can be described with reference to the following clauses.

A method for evaluating an aspirated fluid, the method comprising: receiving a sample cartridge comprising a sample mixture, the sample mixture comprising a liquid biological sample from a subject and a sterilized diluent in an interior volume of the sample cartridge; imaging the sample mixture with a microscopy system; determining whether bacteria present within the sample mixture is above a configurable threshold; and in response to determining that the bacteria within the sample mixture is above the configurable threshold, determining that the liquid biological sample has extracellular bacteria indicative of infection.

The method of any of the other clauses provided herein, wherein imaging the sample mixture comprises imaging the mixture at a z-axis depth of the interior volume positioned above one or more of settled constituents.

The method of any of the other clauses provided herein, further comprising, determining that at least a portion of the bacteria present is from contamination in response to determining the bacteria present in the sample mixture is not above the configurable threshold.

The method of any of the other clauses provided herein, further comprising providing an indication of a likelihood of infection in response to determining the bacteria present in the sample mixture is above the configurable threshold.

The method of any of the other clauses provided herein, wherein the indication of the likelihood of infection is provided on a user interface, a text report, or stored data.

The method of any of the other clauses provided herein, a method of detecting extracellular bacteria, the method comprising: obtaining a liquid biological sample comprising cells from a subject; diluting the liquid biological sample in a sterilized diluent comprising an imaging agent to form a sample mixture; imaging the sample mixture using a microscopy system, wherein the cells remain in a spherical state; and detecting whether extracellular bacteria is present in the liquid biological sample by detecting extracellular bacteria in the sample mixture above a threshold value.

The method of any of the other clauses provided herein, wherein obtaining the liquid biological sample comprises extracting the liquid biological sample from a lesion of the subject.

The method of any of the other clauses provided herein, wherein the lesion is a fluid-filled lesion.

The method of any of the other clauses provided herein, wherein the liquid biological sample is extracted via fine needle aspiration.

The method of any of the other clauses provided herein, wherein diluting the liquid biological sample in the sterilized diluent comprises expelling the liquid biological sample from a sample receptacle via a needle.

The method of any of the other clauses provided herein, wherein the liquid biological sample is expelled into a vial holding the sterilized diluent.

The method of any of the other clauses provided herein, wherein the needle is sterilized prior to expelling the liquid biological sample.

The method of any of the other clauses provided herein, wherein the imaging agent labels DNA.

The method of any of the other clauses provided herein, wherein the imaging agent labels RNA.

The method of any of the other clauses provided herein, wherein the imaging agent is selected from fluorescent dyes, fluorescent stains, fluorescent probes, radioactive labels, quantum dots, labeled antibodies, gram staining, or molecular beacons.

The method of any of the other clauses provided herein, wherein the imaging agent is a fluorescent dye.

The method of any of the other clauses provided herein, wherein the imaging agent is a fluorescent stain.

The method of any of the other clauses provided herein, wherein the threshold value is determined by an expected value of artifactual bacteria.

The method of any of the other clauses provided herein, wherein imaging the sample mixture comprises brightfield microscopy, fluorescent microscopy, or a combination thereof.

The method of any of the other clauses provided herein, wherein the sterilized diluent is housed in a single-use vial.

The method of any of the other clauses provided herein, further comprising detecting a leukocyte in the sample mixture.

The method of any of the other clauses provided herein, wherein the leukocyte is selected from neutrophils, lymphocytes, monocytes, eosinophils, basophil, or a combination thereof.

The method of any of the other clauses provided herein, wherein the neutrophil is a degenerate neutrophil.

The method of any of the other clauses provided herein, wherein the detection of a leukocyte in the sample mixture is indicative of infection.

1. A method for evaluating an aspirated fluid, the method comprising;
receiving a sample cartridge comprising a sample mixture, the sample mixture comprising a liquid biological sample from a subject and a sterilized diluent in an interior volume of the sample cartridge;
imaging the sample mixture with a microscopy system;
determining whether bacteria present within the sample mixture is above a configurable threshold; and
in response to determining that the bacteria within the sample mixture is above the configurable threshold, determining that the liquid biological sample has extracellular bacteria indicative of infection.

2. The method of claim 1, wherein imaging the sample mixture comprises imaging the mixture at a z-axis depth of the interior volume positioned above one or more of settled constituents.

3. The method of claim 1, further comprising, determining that at least a portion of the bacteria present is from contamination in response to determining the bacteria present in the sample mixture is not above the configurable threshold.

4. The method of claim 1, further comprising providing an indication of a likelihood of infection in response to determining the bacteria present in the sample mixture is above the configurable threshold.

5. The method of claim 4, wherein the indication of the likelihood of infection is provided on a user interface, a text report, or stored data.

6. A method of detecting extracellular bacteria, the method comprising:
obtaining a liquid biological sample comprising cells from a subject;
diluting the liquid biological sample in a sterilized diluent comprising an imaging agent to form a sample mixture;
imaging the sample mixture using a microscopy system, wherein the cells remain in a spherical state; and
detecting whether extracellular bacteria is present in the liquid biological sample by detecting extracellular bacteria in the sample mixture above a threshold value.

7. The method of claim 6, wherein obtaining the liquid biological sample comprises extracting the liquid biological sample from a lesion of the subject.

8. The method of claim 7, wherein the lesion is a fluid-filled lesion.

9. The method of claim 6, wherein the liquid biological sample is extracted via fine needle aspiration.

10. The method of claim 6, wherein diluting the liquid biological sample in the sterilized diluent comprises expelling the liquid biological sample from a sample receptacle via a needle.

11. The method of claim 10, wherein the liquid biological sample is expelled into a vial holding the sterilized diluent.

12. The method of claim 10, wherein the needle is sterilized prior to expelling the liquid biological sample.

13. The method of claim 6, wherein the imaging agent labels DNA.

14. The method of claim 6, wherein the imaging agent labels RNA.

15. The method of claim 6, wherein the imaging agent is selected from fluorescent dyes, fluorescent stains, fluorescent probes, radioactive labels, quantum dots, labeled antibodies, gram staining, or molecular beacons.

16. The method of claim 6, wherein the imaging agent is a fluorescent dye.

17. The method of claim 6, wherein the imaging agent is a fluorescent stain.

18. The method of claim 6, wherein the threshold value is determined by an expected value of artifactual bacteria.

19. The method of claim 6, wherein imaging the sample mixture comprises brightfield microscopy, fluorescent microscopy, or a combination thereof.

20. The method of claim 6, wherein the sterilized diluent is housed in a single-use vial.

21. The method of claim 6, further comprising detecting a leukocyte in the sample mixture.

22. The method of claim 21, wherein the leukocyte is selected from neutrophils, lymphocytes, monocytes, eosinophils, basophil, or a combination thereof.

23. The method of claim 22, wherein the neutrophil is a degenerate neutrophil.

24. The method of claim 21, wherein the detection of a leukocyte in the sample mixture is indicative of infection.
